(19) United States
(12) Patent Application Publication
(10) Pub. No.: US 2001/0018194 A1
(43) Pub. Date: Aug. 30, 2001

(54) Luminescence assays

(75) Inventors: Ewald A. Terpetschnig, Sunnyvale, CA (US); John C. Owicki, Palo Alto, CA (US); Sudhir S. Deshpande, Sunnyvale, CA (US)

Correspondence Address: KOLISCH, HARTWELL, DICKINSON, McCORMACK & HEUSER, Suite 200, 520 S.W. Yamhill Street, Portland, OR, 97204, US

(73) Assignee: LJL BioSystems, Inc.

(21) Appl. No.: 09/768,742

(22) Filed: Jan. 23, 2001

Related U.S. Application Data
(63) Continuation of application No. PCT/US99/24707, filed on Oct. 19, 1999.
(60) Provisional application No. 60/104,964, filed on Oct. 20, 1998.
(60) Provisional application No. 60/126,661, filed on Mar. 29, 1999.
(60) Provisional application No. 60/114,209, filed on Dec. 29, 1998.
(60) Provisional application No. 60/116,113, filed on Jan. 15, 1999.
(60) Provisional application No. 60/117,278, filed on Jan. 26, 1999.
(60) Provisional application No. 60/119,884, filed on Feb. 12, 1999.
(60) Provisional application No. 60/121,229, filed on Feb. 23, 1999.
(60) Provisional application No. 60/124,686, filed on Mar. 16, 1999.
(60) Provisional application No. 60/125,346, filed on Mar. 19, 1999.
(60) Provisional application No. 60/130,149, filed on Apr. 20, 1999.
(60) Provisional application No. 60/132,262, filed on May 3, 1999.
(60) Provisional application No. 60/132,263, filed on May 3, 1999.
(60) Provisional application No. 60/135,284, filed on May 21, 1999.
(60) Provisional application No. 60/138,311, filed on Jun. 9, 1999.
(60) Provisional application No. 60/138,438, filed on Jun. 10, 1999.
(60) Provisional application No. 60/138,737, filed on Jun. 11, 1999.
(60) Provisional application No. 60/142,721, filed on Jul. 7, 1999.
(60) Provisional application No. 60/153,251, filed on Sep. 10, 1999.
(60) Provisional application No. 60/138,893, filed on Jun. 11, 1999.

(51) Int. Cl. G01N 33/53; G01N 33/537; G01N 33/543

(52) U.S. Cl. 435/7.92

(57) ABSTRACT

Apparatus, methods, compositions, and kits for improved luminescence assays. These improvements include, among others, the use of mass labeling in luminescence polarization assays, diffusion enhancements in luminescence resonance energy transfer assays, and labeled and/or unlabeled particulates in various luminescence assays.

### CROSS-REFERENCES TO RELATED APPLICATIONS

This application is a continuation of PCT Patent Application Serial No. PCT/US99/24707, filed Oct. 19, 1999, which is incorporated herein by reference.

This application claims priority from the following U.S. Provisional Patent Applications: Serial No. 60/104,964, filed Oct. 20, 1998; and Serial No. 60/126,661, filed Mar. 29, 1999, both of which are incorporated herein by reference.

This application incorporates by reference the following U.S. patent applications: Ser. No. 09/156,318, filed Sep. 18, 1998; and Ser. No. 09/349,733, filed Jul. 8, 1999.

This application also incorporates by reference the following PCT patent applications: Serial No. PCT/US98/23095, filed Oct. 30, 1998; Serial No. PCT/US99/01656, filed Jan. 25, 1999; Serial No. PCT/US99/03678, filed Feb. 19, 1999; Serial No. PCT/US99/08410, filed Apr. 16, 1999; Serial No. PCT/US99/16057, filed Jul. 15, 1999; Serial No. PCT/US99/16453, filed Jul. 21, 1999; Serial No. PCT/US99/16621, filed Jul. 23, 1999; Serial No. PCT/US99/16286, filed Jul. 26, 1999; and Serial No. PCT/US99/16287, filed Jul. 26, 1999.

This application also incorporates by reference the following U.S. provisional patent applications: Serial No. 60/114,209, filed Dec. 29, 1998; Serial No. 60/116,113, filed Jan. 15, 1999; Serial No. 60/117,278, filed Jan. 26, 1999; Serial No. 60/119,884, filed Feb. 12, 1999; Serial No. 60/121,229, filed Feb. 23, 1999; Serial No. 60/124,686, filed Mar. 16, 1999; Serial No. 60/125,346, filed Mar. 19, 1999; Serial No. 60/130,149, filed Apr. 20, 1999; Serial No. 60/132,262, filed May 3, 1999; Serial No. 60/132,263, filed May 3, 1999; Serial No. 60/135,284, filed May 21, 1999; Serial No. 60/138,311, filed Jun. 9, 1999; Serial No. 60/138,438, filed Jun. 10, 1999; Serial No.60/138,737, filed Jun. 11, 1999; Serial No.60/138,893, filed Jun. 11, 1999; Serial No. 60/142,721, filed Jul. 7, 1999; and Serial No. 60/153,251, filed Sep. 10, 1999.

This application also incorporates by reference the following materials: (1) L. Stryer, D. D. Thomas, and C. F. Meares, Diffusion-Enhanced Fluorescence Energy Transfer, 11 Ann. Rev. Biophys. Bioeng. 203 (1982), (2) Max Born and Emil Wolf, Principles of Optics (6^{th} ed. 1980); (3) Richard P. Haugland, Handbook of Fluorescent Probes and Research Chemicals (6^{th} ed. 1996); and (4) Joseph R. Lakowicz, Principles of Fluorescence Spectroscopy (2^{nd} ed. 1983).

### FIELD OF THE INVENTION

The invention relates to luminescence assays. More particularly, the invention relates to improvements in luminescence assays such as luminescence polarization and luminescence resonance energy transfer assays, in some cases involving labeled and/or unlabeled particulates.

### BACKGROUND OF THE INVENTION

Luminescence is the emission of light from excited electronic states of atoms or molecules. Luminescence generally refers to all kinds of light emission, except incandescence, and may include photoluminescence, chemiluminescence, and electrochemiluminescence, among others. In photoluminescence, including fluorescence and phosphorescence, the excited electronic state is created by the absorption of electromagnetic radiation. In chemiluminescence, which includes bioluminescence, the excited electronic state is created by a transfer of chemical energy. In electrochemiluminescence, the excited electronic state is created by an electrochemical process.

Luminescence assays are assays that use luminescence emissions from luminescent analytes to study the properties and environment of the analyte, as well as binding reactions and enzymatic activities involving the analyte, among others. In this sense, the analyte may act as a reporter to provide information about another material or target substance that is the true focus of the assay. Luminescence assays may use various aspects of the luminescence, including its intensity, polarization, lifetime, and sensitivity to energy transfer, among others. Luminescence assays also may use time-independent (steady-state) and/or time-dependent (time-resolved) properties of the luminescence.

Luminescence spectroscopic assays may be based on various luminescence techniques, including fluorescence resonance energy transfer (FRET), fluorescence polarization (FP), fluorescence lifetime (FLT), total internal reflection (TIR) fluorescence, fluorescence correlation spectroscopy (FCS), and fluorescence recovery after photobleaching (FRAP), among others. Each technique has strengths and weaknesses; for example, FRET and FP are especially well suited for assaying binding reactions.

Despite their many uses, luminescence spectroscopic assays suffer from a number of shortcomings. One shortcoming significant in all luminescence assays is the difficulty of labeling the molecule of interest with a fluorophore. This difficulty arises because labeling reactions vary with the label and with the molecule being labeled. For example, the label may not bind in sufficient quantities for detection, or the label may interfere with the biological activity being assayed, or the label may alter the solubility of the compound being labeled, causing it to precipitate.

Another shortcoming significant in many luminescence assays is incomplete spectral separation between the excitation light and emitted luminescence. For example, if a fluorophore has a small Stokes' shift, or if the fluorophore causes significant scattering, it may be difficult or impossible to measure luminescence relative to background.

Another shortcoming significant in many resonance energy transfer assays is incomplete spectral separation of the donor and acceptor. For example, exciting the donor may cause measurable direct excitation of the acceptor, if the excitation spectra of the donor and acceptor overlap sufficiently. Similarly, donor emission may be mistaken for acceptor emission, and vice versa, if the emission spectra of the donor and acceptor overlap sufficiently.

Another shortcoming significant in many resonance energy transfer assays is incomplete temporal separation of the donor and acceptor. For example, the difference in the lifetimes (or apparent lifetimes) of donor and acceptor may not be large enough for the lifetime of either to be resolved.

Insufficient spectral and temporal separation reduces the signal-to-noise ratio and dynamic range of the assay. This reduction is particularly important if one of the labels is present in great excess over the other, or if its luminescence is correspondingly greater.

### SUMMARY OF THE INVENTION

The invention provides apparatus, methods, compositions, and kits for improved luminescence assays. These improvements include, among others, the use of mass labeling in luminescence polarization assays, diffusion enhancements in luminescence resonance energy transfer assays, and labeled and/or unlabeled particulates in various luminescence assays.

### BRIEF DESCRIPTION OF THE DRAWINGS

FIG. 1 is a schematic view of luminescently labeled molecules, showing how molecular reorientation affects luminescence polarization.

FIG. 2 is a schematic view of a frequency-domain time-resolved measurement, showing the definitions of phase angle (phase) φ and demodulation factor (modulation) M.

FIG. 3 is a graph of relative intensity versus wavelength for two luminescent compounds, showing how particulates can be used to enhance luminescence.

FIG. 4 is a graph of phase and modulation versus frequency in a frequency-domain resonance energy transfer experiment, showing how luminescent particulates can be used in luminescence assays.

FIG. 5 is a graph of polarization versus composition, showing how mass labeling can be used to enhance detection of size changes in polarization assays.

FIG. 6 is a graph of polarization versus composition, showing how mass labeling with first and second mass labels can be used to enhance detection of size changes in polarization assays.

FIG. 7 is a graph of polarization versus composition, showing an application of the invention to macromolecules.

### DETAILED DESCRIPTION OF THE INVENTION

The invention provides apparatus, methods, compositions, and kits for performing various assays, including luminescence assays. One aspect of the invention involves using particulates in various luminescence assays, including polarization and resonance energy transfer assays. Another aspect of the invention involves using mass labels to enhance detection of size changes, especially in polarization assays. Yet another aspect of the invention involves using diffusionally mobile donors and/or acceptors in resonance energy transfer assays. These and other aspects of the invention are described in the following sections: (1) luminescence assays, and (2) description of methods, compositions, and kits.

### 1. Luminescence Assays

Luminescence is the emission of light from excited electronic states of atoms or molecules. As described below, luminescence may be used in a variety of assays, including (A) intensity assays, (B) polarization assays, (C) energy transfer assays, and (D) time-resolved assays.

A. Intensity Assays

Luminescence intensity assays involve monitoring the intensity (or amount) of light emitted from a composition. The intensity of emitted light will depend on the extinction coefficient, quantum yield, and number of luminescent analytes in the composition, among others. These quantities, in turn, will depend on the environment of the analyte, among others, including the proximity and efficacy of quenchers and energy transfer partners. Thus, luminescence intensity assays may be used to study binding reactions, among other applications.

B. Polarization Assays

Luminescence polarization assays involve the absorption and emission of polarized light, and typically are used to study molecular rotation. (Polarization describes the direction of light's electric field, which generally is perpendicular to the direction of light's propagation.)

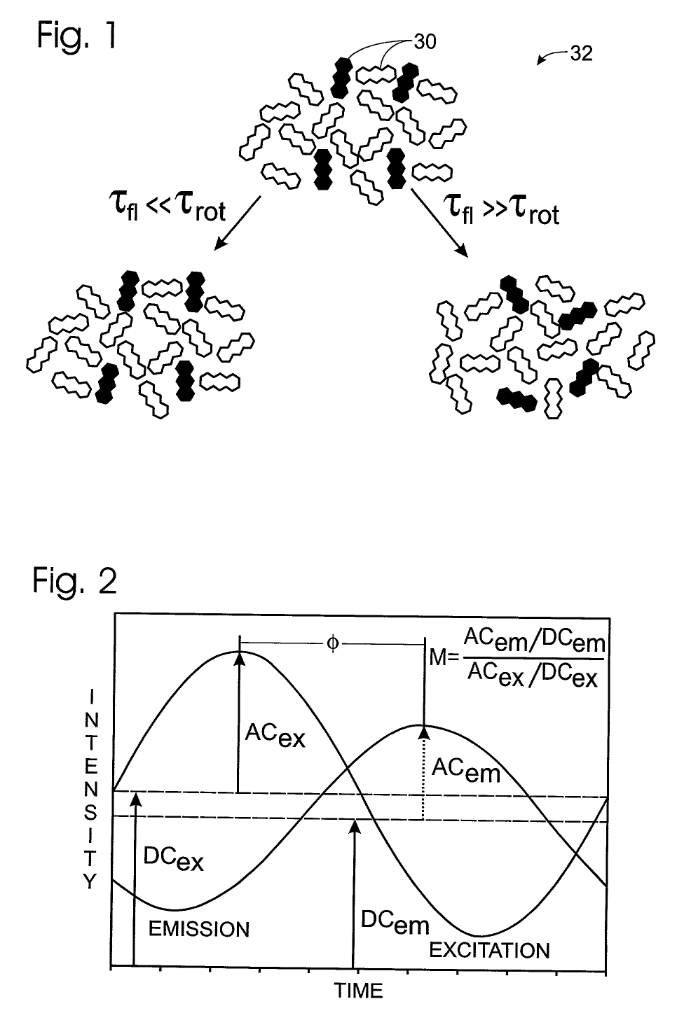

FIG. 1 is a schematic view showing how luminescence polarization is affected by molecular rotation. In a luminescence polarization assay, specific molecules 30 within a composition 32 are labeled with one or more luminophores. The composition then is illuminated with polarized excitation light, which preferentially excites luminophores having absorption dipoles aligned parallel to the polarization of the excitation light. These molecules subsequently decay by preferentially emitting light polarized parallel to their emission dipoles. The extent to which the total emitted light is polarized depends on the extent of molecular reorientation during the time interval between luminescence excitation and emission, which is termed the luminescence lifetime, τ. The extent of molecular reorientation in turn depends on the luminescence lifetime and the size, shape, and environment of the reorienting molecule. Thus, luminescence polarization assays may be used to quantify binding reactions and enzymatic activity, among other applications. In particular, molecules commonly rotate via diffusion with a rotational correlation time τᵣₒₜ that is proportional to their size. Thus, during their luminescence lifetime, relatively large molecules will not reorient significantly, so that their total luminescence will be relatively polarized. In contrast, during the same time interval, relatively small molecules will reorient significantly, so that their total luminescence will be relatively unpolarized.

The relationship between polarization and intensity is expressed by the following equation:

$$P = \frac{{I}_{∥} - {I}_{⊥}}{{I}_{∥} + {I}_{⊥}} \qquad ( 1 )$$

Here, P is the polarization, I_{∥} is the intensity of luminescence polarized parallel to the polarization of the excitation light, and I_{⊥} is the intensity of luminescence polarized perpendicular to the polarization of the excitation light. P generally varies from zero to one-half for randomly oriented molecules (and zero to one for aligned molecules). If there is little rotation between excitation and emission, I_{∥} will be relatively large, I_{⊥} will be relatively small, and P will be close to one-half. (P may be less than one-half even if there is no rotation; for example, P will be less than one if the absorption and emission dipoles are not parallel.) In contrast, if there is significant rotation between absorption and emission, I_{∥} will be comparable to I_{⊥}, and P will be close to zero. Polarization often is reported in milli-P (mP) units (1000×P), which for randomly oriented molecules will range between 0 and 500, because P will range between zero and one-half.

Polarization also may be described using other equivalent quantities, such as anisotropy. The relationship between anisotropy and intensity is expressed by the following equation:

$$r = \frac{{I}_{∥} - {I}_{⊥}}{{I}_{∥} + 2 {I}_{⊥}} \qquad ( 2 )$$

Here, r is the anisotropy. Polarization and anisotropy include the same information, although anisotropy may be more simply expressed for systems containing more than one luminophore. In the description and claims that follow, these terms may be used interchangeably, and a generic reference to one should be understood to imply a generic reference to the other.

The relationship between polarization, luminescence lifetime, and rotational correlation time is expressed by the Perrin equation:

$$( \frac{1}{P} - \frac{1}{3} ) = ( \frac{1}{{P}_{0}} - \frac{1}{3} ) \cdot ( 1 + \frac{τ}{{τ}_{rot}} ) \qquad ( 3 )$$

Here, P₀ is the polarization in the absence of molecular motion (intrinsic polarization), τ is the luminescence lifetime (inverse decay rate) as described above, and τᵣₒₜ is the rotational correlation time (inverse rotational rate) as described above.

The Perrin equation shows that luminescence polarization assays are most sensitive when the luminescence lifetime and the rotational correlation time are similar. Rotational correlation time is proportional to molecular weight, increasing by about 1 nanosecond for each 2,400 Dalton increase in molecular weight (for a spherical molecule). For shorter lifetime luminophores, such as fluorescein, which has a luminescence lifetime of roughly 4 nanoseconds, luminescence polarization assays are most sensitive for molecular weights less than about 40,000 Daltons. For longer lifetime probes, such as Ru(bpy)₂dcbpy (ruthenium 2,2′-dibipyridyl 4,4′-dicarboxyl-2,2′-bipyridine), which has a lifetime of roughly 400 nanoseconds, luminescence polarization assays are most sensitive for molecular weights between about 70,000 Daltons and 4,000,000 Daltons.

C. Energy Transfer Assays

Energy transfer is the transfer of luminescence energy from a donor luminophore to an acceptor without emission by the donor. In energy transfer assays, a donor luminophore is excited from a ground state into an excited state by absorption of a photon. If the donor luminophore is sufficiently close to an acceptor, excited-state energy may be transferred from the donor to the acceptor, causing donor luminescence to decrease and acceptor luminescence to increase (if the acceptor is luminescent). The efficiency of this transfer is very sensitive to the separation R between donor and acceptor, decaying as 1/R⁻⁶. Energy transfer assays use energy transfer to monitor the proximity of donor and acceptor, which in turn may be used to monitor the presence or activity of an analyte, among others.

Energy transfer assays may focus on an increase in energy transfer as donor and acceptor are brought into proximity. These assays may be used to monitor binding, as between two molecules X and Y to form a complex X:Y. Here, colon (:) represents a noncovalent interaction. In these assays, one molecule is labeled with a donor D, and the other molecule is labeled with an acceptor A, such that the interaction between X and Y is not altered appreciably. Independently, D and A may be covalently attached to X and Y, or covalently attached to binding partners of X and Y.

Energy transfer assays also may focus on a decrease in energy transfer as donor and acceptor are separated. These assays may be used to monitor cleavage, as by hydrolytic digestion of doubly labeled substrates (peptides, nucleic acids). In one application, two portions of a polypeptide are labeled with D and A, so that cleavage of the polypeptide by a protease such as an endopeptidase will separate D and A and thereby reduce energy transfer. In another application, two portions of a nucleic acid are labeled with D and A, so that cleave by a nuclease such as a restriction enzyme will separate D and A and thereby reduce energy transfer.

Energy transfer between D and A may be monitored in various ways. For example, energy transfer may be monitored by observing an energy-transfer induced decrease in the emission intensity of D and increase in the emission intensity of A (if A is a luminophore). Energy transfer also may be monitored by observing an energy-transfer induced decrease in the lifetime of D and increase in the apparent lifetime of A.

In a preferred mode, a long-lifetime luminophore is used as a donor, and a short-lifetime luminophore is used as an acceptor. Suitable long-lifetime luminophores include metal-ligand complexes containing ruthenium, osmium, etc., and lanthanide chelates containing europium, terbium, etc. In time-gated assays, the donor is excited using a flash of light having a wavelength near the excitation maximum of D. Next, there is a brief wait, so that electronic transients and/or short-lifetime background luminescence can decay. Finally, donor and/or acceptor luminescence intensity is detected and integrated. In frequency-domain assays, the donor is excited using time-modulated light, and the phase and/or modulation of the donor and/or acceptor emission is monitored relative to the phase and/or modulation of the excitation light. In both assays, donor luminescence is reduced if there is energy transfer, and acceptor luminescence is observed only if there is energy transfer.

D. Time-Resolved Assays

Time-resolved assays involve measuring the time course of luminescence emission. Time-resolved assays may be conducted in the time domain or in the frequency domain, both of which are functionally equivalent.

In a time-domain measurement, the time course of luminescence is monitored directly. Typically, a composition containing a luminescent analyte is illuminated using a narrow pulse of light, and the time dependence of the intensity of the resulting luminescence emission is observed, although other protocols also may be used. For a simple molecule, the luminescence commonly follows a single-exponential decay.

In a frequency-domain measurement, the time course of luminescence is monitored indirectly, in frequency space. Typically, the composition is illuminated using light whose intensity is modulated sinusoidally at a single modulation frequency f, although other protocols (such as transforming time-domain data into the frequency domain) also may be used. The intensity of the resulting luminescence emission is modulated at the same frequency as the excitation light. However, the emission will lag the excitation by a phase angle (phase) φ, and the intensity of the emission will be demodulated relative to the intensity of the excitation by a demodulation factor (modulation) M.

FIG. 2 shows the relationship between emission and excitation in a single-frequency frequency-domain experiment. The phase φ is the phase difference between the excitation and emission. The modulation M is the ratio of the AC amplitude to the DC offset for the emission, relative to the ratio of the AC amplitude to the DC offset for the excitation. The phase and modulation are related to the luminescence lifetime τ by the following equations:

ωτ=tan (φ) (4)

$$ωτ = \sqrt{\frac{1}{{M}^{2}} - 1} \qquad ( 5 )$$

Here ω is the angular modulation frequency, which equals 2π times the modulation frequency. For maximum sensitivity, the angular modulation frequency should be roughly the inverse of the luminescence lifetime. Lifetimes of interest in high-throughput screening vary from less than 1 nanosecond to greater than 1 millisecond. Therefore, instruments for high-throughput screening should be able to cover modulation frequencies from less than about 200 Hz to greater than about 200 MHz.

### 2. Description of Methods, Compositions, and Kits

The invention provides methods, compositions, and kits for improved luminescence assays. These methods, compositions, and kits may be practiced using apparatus, methods, and compositions described in the above-identified patent applications, which are incorporated herein by reference. For example, luminescence may be detected using high-sensitivity luminescence apparatus, including those described in U.S. patent application Ser. No. 09/062,472, filed Apr. 17, 1998, U.S. patent application Ser. No. 09/160,533, filed Sep. 24, 1998, and PCT Patent Application Serial No. PCT/US98/23095, filed Oct. 30, 1998. Luminescence also may be detected using high-sensitivity luminescence methods, including those described in PCT Patent Application Serial No. PCT/US99/01656, filed Jan. 25, 1999, and PCT Application Serial No. PCT/US99/03678, filed Feb. 19, 1999. Luminescence also may be detected using sample holders optimized for performance with the above-identified high-sensitivity luminescence apparatus and methods, including those described in PCT Patent Application Serial No. PCT/US99/08410, filed Apr. 16, 1999.

A. Particulate-Based Assays

The invention provides methods, compositions, and kits relating to use of particulates in luminescence assays, particularly luminescence polarization assays and luminescence resonance energy transfer assays.

Labeled particulates may be formed by associating luminophores or chromophores with particulates, such as macromolecules, dendrimers, beads (e.g., glass, latex, or polyacrylnitrile), or liposomes, among others. Depending on the particulate, labels may be associated covalently or entrapped noncovalently, such as through electrostatic interactions. For example, macromolecules and dendrimers can be labeled by incorporating the label into the compound during synthesis, or by trapping the label in pockets, among other mechanisms. Beads may be labeled by incorporating the label into the bead during formation, among other mechanisms. Liposomes may be labeled by attaching the label to the lipid membrane of the liposome (e.g., using a hydrophilic tether having a hydrophobic tail inserted into the bilayer) or by trapping the label within the lumen of the liposome, among other mechanisms. Incorporation of the label in a particulate may reduce the variability that results from attaching the label directly to the molecule of interest. Incorporating the label into a particulate also may enhance luminescence, facilitating detection, by reducing accessibility to oxygen and/or because many labels may be attached to a single particulate. For example, incorporating lanthanides into particulates should reduce collisional and static quenching, which often are manifest as negative side effects in assays involving lanthanides.

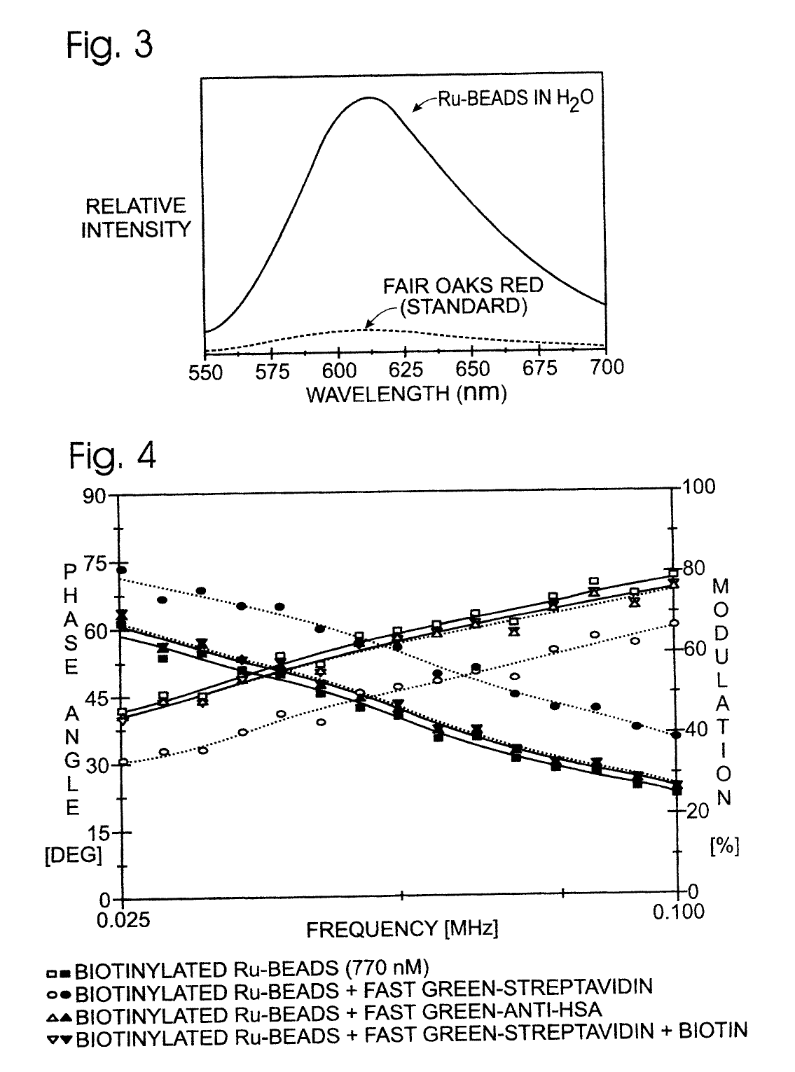

FIG. 3 shows how luminescence may be enhanced by encapsulating a luminophore in a particulate. Specifically, this figure shows relative intensities of Fair Oaks Red™ (5-isothiocyanato-1,10-phenanthroline-bis(2,2′-bipyridine) ruthenium(II) hexafluorophosphate) in aqueous solution and a solution of Ru-tris-bathophenanthroline (tris(bathophenanthroline) ruthenium(II) hexafluorophosphate) encapsulated in beads. These compounds are shown below:

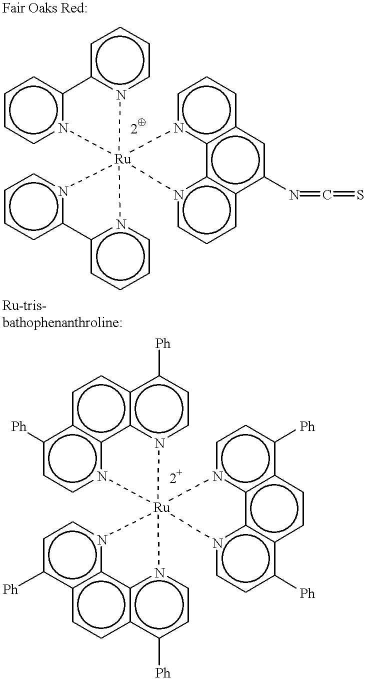

The luminescence of the Ru-tris-bathophenanthroline significantly exceeds the luminescence of the Fair Oaks Red, even though the quantum yield of the former compound is similar to the quantum yield of the latter compound in aqueous solution. This increase in luminescence upon encapsulation arises in part because the encapsulated compound is protected from oxygen, which quenches luminescence.

Unlabeled particulates may include colloidal gold and semiconductor nanocrystals, among others. Semiconductor nanocrystals also are known as quantum dots. Colloidal gold and semiconductor nanocrystals are especially useful in energy transfer assays, as described below.

Particulates may be attached covalently or noncovalently to a molecule of interest to perform an assay; such molecules of interest may include biomolecules, drugs, polymers, and other molecules. Particulates may be attached covalently by adding reactive groups to the surface of the particulate, and then allowing these reactive groups to react covalently with the molecule of interest. Suitable reactive groups include alkyl-carboxyl, amino, hydroxyl, N-hydroxysuccinimid-ester, isothiocyanate, maleimide, sulfonxyl, and thiol functions, among others. Particulates may be attached noncovalently by using biotin/streptavidin, antibody/antigen, and lectin (e.g., concanavalin A)/sugar, among others. For example, the molecule of interest could be biotinylated and incubated with particulate-labeled streptavidin. Alternatively, the molecule of interest could be incubated with particulate-labeled antibodies against the molecule of interest or against a hapten attached to the molecule of interest.

Particulates may incorporate almost any luminophore or other label, maximizing their spectral and temporal flexibility. Spectral flexibility is important in all luminescence assays, and especially in energy transfer assays where pairs of labels must be employed. Temporal flexibility is especially important in polarization assays, where luminescence lifetime and rotational correlation time may be matched to improve resolution. For example, particulates may include metal-ligand complexes or lanthanides as luminophores, which have long lifetimes (several hundred nanoseconds to milliseconds) and large Stokes' shifts.

Particulates also may have almost any size and shape. Diameters may range from about one to several nanometers to many microns. Small sizes reduce scattering and hence background, making small particulates especially attractive for luminescence assays. Small sizes are appropriate for most energy transfer assays, because if the diameter of the donor-labeled particulate greatly exceeds the R₀ distance for efficient energy transfer (3-10 nm), much donor luminescence will not participate in energy transfer. A range of sizes is appropriate for polarization assays, because a range permits the rotational correlation time of the particulate to be matched to the lifetime of the luminophore, as described below.

Energy transfer assays.

Labeled particulates may improve energy transfer assays in a variety of ways. Particulates may be easier to label than the binding partners of interest. Particulates also may increase the number of acceptors available for energy transfer from a given donor, or vice versa, enhancing energy transfer. Particulates such as colloidal gold may act as acceptors (quenchers), without themselves contributing luminescence that may be mistaken for donor luminescence. Particulates such as semiconductor nanocrystals may act as donors in energy transfer assays, with absorption and emission properties as well as luminescence lifetimes that can be engineered by controlling size and composition.

FIG. 4 shows an application of the invention to energy transfer between biotinylated Ru-tris-bathophenanthroline beads (Ru-beads; τ˜7 μs) and Fast Green™-streptavidin (FG-SA). Ru-beads were described above; Fast Green is shown below:

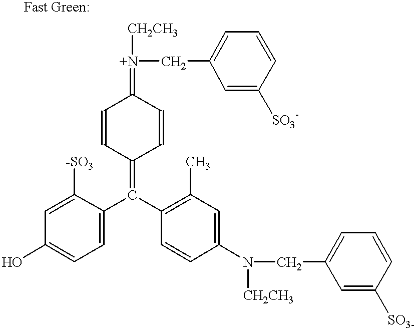

These experiments were performed in a microplate, using a reference well and four sample wells. The reference well included 100 μL of a 1 nM fluorescein solution. The sample wells included (1) 770 μM biotinylated Ru-beads, (2) 770 μM biotinylated Ru-beads and 16 μM FG-SA (D/P ratio=3.4:1), (3) 770 μM biotinylated Ru-beads and 16 μM Fast Green-labeled anti-HSA (D/P ratio=1.2:1), and (4) 770 μM biotinylated Ru-beads, 16 μM FG-SA, and a tenfold excess of biotin (relative to the streptavidin). In well (4), the FG-SA was pre-incubated with the biotin prior to addition of the biotinylated Ru-beads. After preparation, wells were mixed at room temperature for 30 minutes, and frequency responses were then measured between 0.025 MHz and 0.1 MHz using an LJL FLARE™ microplate reader and a fluorescein reference having a 4-nanosecond lifetime. FIG. 4 shows (phase and modulation) frequency response curves for specific binding between the biotinylated Ru-beads and the FG-SA. The frequency response curve of the Ru-beads is shifted to higher frequencies in the presence of FG-SA due to energy transfer. This energy transfer is reversed by incubation of FG-SA with a tenfold excess of biotin prior to addition of the biotinylated Ru-beads. The control experiment with acceptor-labeled anti-HSA did not result in considerable changes in phase angle or modulation.

Polarization assays.

Labeled particulates may improve polarization assays in a variety of ways. Particulates may be labeled with multiple luminophores, enhancing signal. Particulates also may be labeled with long-lifetime luminophores, permitting detection of slow rotational motions. For example, particulates labeled with long-lifetime luminophores such as metal-ligand complexes are ideally suited to measuring binding to membranes or membrane receptors and to measuring agglutination reactions.

The polarization assays provided by the invention work best if the luminescent label maintains a fixed orientation relative to the labeled molecule, so that depolarization reflects reorientation of the labeled molecule and not merely reorientation of the label relative to the labeled molecule. For this reason, particulates for polarization assays are best labeled so that the luminophores are immobilized within the particulate. Suitable particulates include small glass, latex beads, hydrophilic functionalized macromolecules, and dentrimers.

The polarization assays provided by the invention also work best if the lifetime of the luminophore is long relative to the rotational correlation time of the unbound particulate and short relative to the rotational correlation time of the bound particulate. Under these conditions, luminescence emitted by the labeled particulate is unpolarized if the particulate is unbound and polarized if the particulate is bound.

A further advantage of particulate based polarization assays lies in the potential multivalency of the particle. That is, it often is possible to attach multiple copies of a molecule of interest, such as a ligand, to the particle. In polarization assays for the interaction between a ligand and a membrane-bound receptor, insufficient affinity of the labeled ligand for the receptor often is a limiting factor in assay performance. A multivalent ligand-labeled particle helps overcome this problem in two ways. First, the mere presence of multiple ligands on each labeled structure increases the affinity of the labeled structure for the receptor to an extent approximately proportional to the degree of multivalency. Second, receptors generally are presented embedded in membrane fragments, with many copies of receptors per membrane fragment. Thus, the membrane fragments are multivalent with respect to receptors, and the combination of multivalent ligand structures and multivalent receptor structures can be expected to lead to increases in the effective affinity of the interaction, by analogy to the avidity effects that are recognized in immunological interactions involving multivalent antibodies and multivalent antigens.

B. Mass-Labeling Assays

The invention also provides methods and kits for detecting an analyte in a sample. These methods and kits may involve a probe and a mass label for changing the effective size of the probe. For example, the methods may include the following steps: (1) forming a complex including first, second, and third members, where the first member is a probe, the second member is a mass label, and the third member is selected from the group consisting of the analyte, a compound that specifically binds to the analyte, and a product formed by the analyte, wherein no significant binding occurs involving the first and second members in the absence of the third member, (2) measuring a property of the probe that is sensitive to the size of the complex, (3) correlating the property with the presence or activity of analyte in the sample.

The invention may be used with a variety of probes and a variety of assays. Suitable combinations of probes and assays include (1) luminophores and luminescence assays, such as luminescence polarization assays, (2) scatterers and light-scattering assays, such as dynamic light scattering (DLS) assays, and (3) spin probes and magnetic resonance assays, such as electron spin resonance (ESR) or electron paramagnetic resonance (EPR) assays.

The utility of the invention is easily understood in the context of a luminescence polarization experiment. In conventional polarization assays, a probe is labeled with a luminophore selected to have a luminescence lifetime comparable to or longer than the rotational correlation time of the labeled molecule in the free state but shorter than the rotational correlation time of the labeled molecule in the bound state. In many cases, finding such a luminophore may be difficult, especially if there are limitations on both its spectrum and its lifetime. In polarization assays provided by the invention, the mass of the binding partner of the probe may be modified so that the rotational correlation time of the probe in the bound state exceeds the lifetime of the luminophore. This is accomplished by “mass-labeling” the binding partner, for example, by binding of particulates or antibodies, among others. In this way, the rotational correlation time may be adjusted to complement the luminescence lifetime, rather than the luminescence lifetime being selected to complement the rotational correlation time.

Following is a schematic description of some of the types of fluorescence-polarization assays in which mass weighting can increase the amplitude of the signal. Dashes (-) indicate covalent bonds, and colons (:) indicate biologically specific noncovalent interactions.

A first type of mass-label fluorescence-polarization assay seeks to measure the presence of inhibition of the binding interaction of two molecules X and Y (forming X:Y) by some putative inhibitor I present in a screening library. Perhaps I competes with X for a binding site on Y or allosterically weakens the binding of X to Y. In the assay, a fluorophore F is covalently labeled to X in a way that does not interfere with the formation of X:Y, and F-X is incubated with Y and I under conditions where an appreciable amount of F-X:Y is formed if I is an ineffective inhibitor but detectably less F-X:Y is formed if I does show significant inhibitory potency. Presumably, the rotational correlation time of F-X is shorter than the lifetime of F, so that the emission from F is substantially depolarized. If the rotational correlation time of F-X:Y is still not much longer than the lifetime of F, the polarization increase upon the binding of F-X to Y will not be great. If, however, a mass label for Y is introduced such that the complex F-X:Y:M can be formed, the rotational correlation of the bound F-X will be increased, as will the change in polarization upon binding. If M is multivalent for Y, then the mass of the complex will be further increased. If a second mass label N is introduced that binds either to a distinct site on Y or to M, further increases in mass and polarization change can be effected. Multivalency of N may have beneficial effects due to cross-linking.

A second type of mass-label fluorescence-polarization assay can be used to test the ability of a compound to inhibit or enhance enzymatic activity. The assay comprises a fluorescently labeled enzyme substrate F-S that does not bind a second biological molecule Y until it is converted enzymatically into the product F-P (i.e., F-P:Y forms, but not F-S:Y). The signal in the assay hinges on the difference in polarization between F-S and F-P:Y. The same mass-labeling considerations apply to F-P:Y that applied to F-X:Y in the discussion above.

A third type of mass-label fluorescence-polarization assay also can be used to test the ability of a compound to inhibit or enhance enzymatic activity, in cases where the enzyme cleaves substrates such as oligo- or polypeptides, oligo- or polysaccharides, oligo- or polynucleotides, and non-nucleotide phosphodiesters. The requirement is that the enzyme cleave the substrate into two separate molecules or cause the dissociation of a tight non-covalent interaction, and that the fluorophore label be on one fragment while a biological recognition site for binding to another molecule be on the other fragment.

For example, consider the substrate A-B, which the enzyme cleaves into A and B. Label the A component with a fluorophore F and the B component with a moiety X that binds to Y, forming F-A-B-X or F-A-B-X:Y, depending on the presence of Y. To the extent that the enzyme is active, the fragments F-A and B-X will be formed, so that the rotational correlation time of F will not be increased in the presence of Y (i.e., only B-X:Y will be formed rather than F-A-B-X:Y). The same mass-labeling considerations apply to F-A-B-X:Y that applied to F-P:Y and F-X:Y in the preceding discussions.

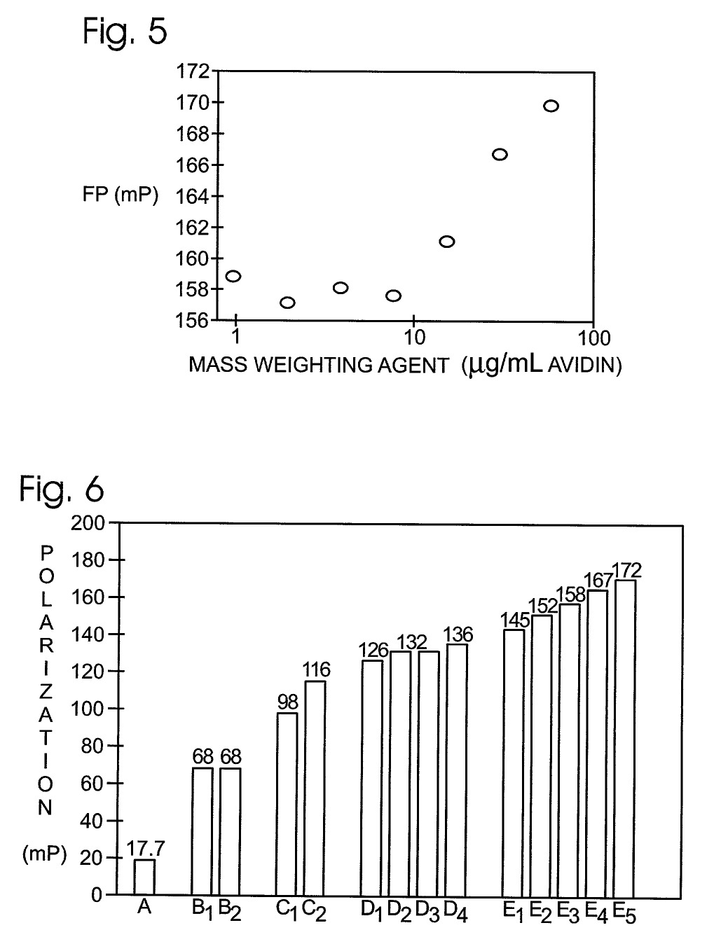

FIG. 5 shows an application of the invention to the detection of an antibody using luminescence polarization. Here the analyte is a biotinylated rabbit anti-bovine gamma globulin (BGG) antibody, the probe is a BGG (principally IgG) antibody, and the mass label is avidin, a tetravalent biotin-binding protein having a mass of about 60 kDa. This application is a setup for an assay for compounds that inhibit the binding of BGG to anti-BGG. The probe is labeled at a concentration of about 8 luminophores per protein with Sunnyvale Red™ isothiocyanate ([4,4′-Bis [(2-isothiocyanato)ethoxycarbonyl)]-2,2′bipyridine]bis(2,2′-bipyridine)ruthenium(II)hexafluorophosphate), which is shown below:

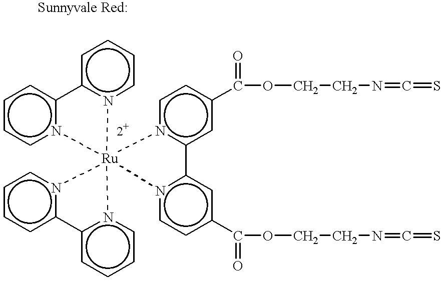

The polarization of the probe alone in phosphate buffered saline at about 16 μg/mL is about 86 mP. The polarization of the probe after it is contacted with the analyte at about 17 μg/mL is about 152 mP. The polarization of the probe after addition of the mass label is about 157 to 170 mP, depending on the concentration of mass label. The mass label increases the mass of the bound complex both by adding its own mass and by cross-linking the immune complexes.

FIG. 6 shows an application of the invention to the detection of a serum protein using luminescence polarization. Here the analyte is biotinylated bovine serum albumin (BSA), the probe is streptavidin (SA) labeled with Sunnyvale Red isothiocyanate, and the mass label is either a rabbit anti-BSA antibody or the rabbit anti-BSA antibody and a goat anti-rabbit IgG antibody. The goat antibody functions as both a secondary mass label and a crosslinker to crosslink probes. This application is a setup for an assay for compounds that inhibit the binding of biotin to streptavidin. The figure shows five sets of experiments, which involve measurement of the polarization from (A) the probe alone, (B) the probe and analyte, (C) the probe, analyte, and mass label, and (D, E) the probe, analyte, mass label, and secondary mass label/crosslinker. The overall change in polarization from A to E is about 154 mP, corresponding to about a tenfold increase. The experimental conditions are summarized in the following table, where the numbers indicate molar ratios:

| | SA-RuMLC | | | |
|---|---|---|---|---|
| Sample | [25 μg/mL] | Biotin-BSA | Anti-BSA | Anti-Rabbit |
| A | 1 | | | |
| B₁ | 1 | 1 | | |
| B₂ | 1 | 2 | | |
| C₁ | 1 | 2 | 2 | |
| C₂ | 1 | 2 | 4 | 4 |
| D₂ | 1 | 2 | 4 | 8 |
| D₃ | 1 | 2 | 4 | 12 |
| D₄ | 1 | 2 | 4 | 16 |
| E₁ | 1 | 2 | 8 | 16 |
| E₂ | 1 | 2 | 16 | 16 |
| E₃ | 1 | 2 | 24 | 16 |
| E₄ | 1 | 2 | 32 | 16 |
| E₅ | 1 | 2 | 40 | 16 |

The methods and kits provided by the invention generally involve an analyte sample, probe, and mass label.

The analyte generally includes any species capable of specifically binding to a probe. In most applications, the analyte will be a molecular or supermolecular species, such as a biomolecule. The analyte may be native to the sample, added to the sample, or created within the sample, such as by a reaction. If the analyte is created within the sample, the invention may be used to correlate the presence and/or amount of the analyte in the sample with the presence or activity of another compound, such as an enzyme. In general, the analyte may be a species to be quantified, as in diagnostics, or a species whose activity is to be quantified, as in drug screening.

Specific binding means binding to the specific binding partner to the exclusion of binding to most other moieties. Specific binding can be characterized by a binding coefficient. Generally, specific binding coefficients range from 10⁻⁴ M to 10⁻¹² M and lower, and preferred specific binding coefficients range from 10⁻⁹ M to 10⁻¹² M or lower. Generally, fragments, derivatives, or analogs of specific binding partners also may be used, if such fragments, derivatives, and analogs retain their specificity and binding affinity for their binding partners.

The sample generally comprises any composition for which the presence or activity of analyte is to be tested. The sample may be natural, artificial, or a combination thereof. Suitable samples include or may be derived from compounds, mixtures, surfaces, solutions, emulsions, suspensions, cell cultures, fermentation cultures, cells, tissues, secretions, and/or derivatives and/or extracts thereof, among other compositions.

The probe generally includes any species capable of specifically binding to a member selected from the group consisting of the analyte, a compound that specifically binds to the analyte, and a product formed by the analyte. Thus, the probe may be selected based on its ability to bind to the member, and the probe and member together constitute specific binding partners. Typically, binding between the probe and member is noncovalent. Suitable combinations of probes and members (or members and probes) include immunological binding partner/antigen, biotin/avidin, and lectin (e.g., concanavalin A)/sugar, among others. Suitable immunological binding partners include polyclonal and monoclonal antibodies. Immunological binding partners also include chimeric, single chain, and humanized antibodies, as well as Fab fragments and the products of Fab expression libraries. The probe may include one or more components, where at least one of the components is capable of specifically binding to the analyte. A one-component probe might be ethidium bromide, for binding to nucleic acid analytes, while a two component probe might be a photoluminescent particulate associated with an antibody, for binding to corresponding antigenic analytes. Generally, the probe will have a measurable or detectable property that is sensitive to the size and particularly the dynamics of the complex formed by binding of the probe, analyte, and mass label.

In some applications, the probe may be photoluminescent, so that the measurable property relates to photoluminescence. Such applications include photoluminescence polarization experiments, as show in FIGS. 5 and 6. In polarization experiments, detection of analyte will be improved if the probe and mass label are selected such that photoluminescence lifetime of the probe is greater than the rotational correlation time of the unbound probe and less than the rotational correlation time of the complex formed by binding of the probe, member, and mass label. In particular, detection will be significantly improved if the difference in polarization of free and complexed probe is greater than about 100 mP.

The mass label generally includes any species capable of specifically binding to the member or to a complex formed by binding of the probe to the member, but not to the probe alone. Thus, like the probe, the mass label may be selected based on its ability to bind to the member. However, the probe also is selected in part for its measurable or detectable properties relating to binding to the member, whereas the mass label is selected in part for its ability to effect changes in the effective size and/or dynamics of the complexed probe. Suitable mass labels include immunological binding partners and particulates associated with immunological binding partners, among others.

Ideally, the size and/or mass of the mass label will be significant relative to the complex formed by binding of the probe to the member, so that the mass label will appreciably and detectably alter a measurable property of the complex. This is especially important for particulate probes, which may require binding by especially large and/or especially large numbers of mass labels to be measurably or detectably slowed. In some embodiments, the mass label may be capable of specifically binding to more than one member, so that the mass label may crosslink at least two members and the associated probes, further increasing the mass. In other embodiments, the mass labels may include target groups capable of interacting with secondary mass labels or crosslinkers. Such target groups may be intrinsic to the mass label, such as immunological binding sites, or extrinsic to the mass label, such as conjugated biotin, avidin, lectin, and sugar, among others. The secondary mass labels or crosslinkers may be capable of specifically binding to the member, the complex formed by binding of the probe to the member, or another mass label. The probe and mass label may be selected based on binding affinities such that the average number of mass labels bound to member exceeds the average number of probes bound to member.

The sample may be contacted with the probe and/or mass label using any method for effectuating such contact. A preferred method is by adding the probe and/or mass label to the sample, or mixing the materials in solution, although other methods also may be used.

The methods provided by the invention may involve measurement of any property that is sensitive to the size and/or the dynamics of the complex formed by binding of the probe, mass label, and member. Such properties may include rotational and/or translational diffusion coefficients. Generally, the rotational diffusion coefficient provides a more sensitive measure of size and dynamics than the translational diffusion coefficient. This is because the rotational diffusion coefficient varies inversely with the volume (i.e., radius cubed) of the diffusing species, whereas the translational diffusion coefficient varies inversely with the radius of the diffusing species.

In some applications, the invention may be used to detect analytes in successive samples. Such applications may involve repeating on first and second samples the steps of forming a complex, measuring a property of the probe, and correlating the property with the presence or activity of analyte. Such applications also may involve comparing the amounts of analyte in the first and second samples and/or correlating the amounts with the presence or activity of another compound.

C. Diffusion-Enhanced Luminescence Resonance Energy Transfer Assays

The invention also provides methods and kits for performing a resonance energy transfer assay using a donor/acceptor energy transfer pair. These methods and kits may be constructed such that one or both members of a energy transfer pair are diffusionally mobile. Here diffusion and diffusionally mobile are broadly defined to include any kind of random thermal motion, even if constrained, as by a tether or interface. Diffusional mobility, in turn, may increase the number of acceptors favorably positioned for energy transfer from a given donor, or vice versa, enhancing energy transfer. This process can be termed diffusion-enhanced resonance energy transfer (DE-RET). The methods may include the following steps: (1) providing first and second members of a donor/acceptor pair, the pair being capable of resonance energy transfer, (2) binding the first member to a binding partner, (3) permitting the first member to be diffusionally mobile relative to the binding partner while it is bound to the binding partner, and (4) detecting a change in proximity between the first and second member.

Generally speaking, diffusion will enhance RET if the diffusion causes a significant increase in the probability that an acceptor will appear within efficient energy-transfer range of a donor during the lifetime of the donor (i.e., roughly within the R₀ distance at which energy transfer becomes 50% probable during a lifetime). Because the probability of a diffusional collision between donor and acceptor increases with time, the longer the lifetime of the donor, the greater the potential enhancement of energy transfer due to diffusion.

This argument can be quantified, as has been reviewed by Stryer, Thomas, and Meares (1982). We proceed less formally here. Diffusion can be described using the diffusion equation, which states that [symbol] r² [symbol] =2nDt, where [symbol] r² [symbol] is the mean-square distance diffused, n is the dimensionality (1, 2, or 3) of the diffusion process, D is the diffusion coefficient of acceptor relative to donor (assuming that the acceptor and donor are not linked rigidly to each other), and t is the time of diffusion.

The following table shows the RMS distance diffused rᵣₘₛ (=\sqrt{ [symbol]r² [symbol]}=\sqrt{4Dτ}) for n=2 (corresponding to surface diffusion) and a typical range of diffusion coefficients and fluorescence lifetimes (τ, serving as the time t during which diffusion takes place). The lifetimes are 5 nanoseconds (ns, typical of xanthene dyes), 500 ns (typical of Ru, Os, and Re metal-ligand charge-transfer complexes), and 500 microseconds (μs, typical of lanthanide chelates and cryptates).

| | | | |
|---|---|---|---|
| D (cmˆ 2/s) = | 1.0E−06 | 1.0E−06 | 1.0E−06 |
| tau (s) = | 5.0E−09 | 5.0E−07 | 5.0E−04 |
| r (nm) = | 1.4E+00 | 1.4E+01 | 4.5E+02 |
| D (cmˆ 2/s) = | 1.0E−07 | 1.0E−07 | 1.0E−07 |
| tau (s) = | 5.0E−09 | 5.0E−07 | 5.0E−04 |
| r (nm) = | 4.5E−01 | 4.5E+00 | 1.4E+02 |
| D (cmˆ 2/s) = | 1.0E−08 | 1.0E−08 | 1.0E−08 |
| tau (s) = | 5.0E−09 | 5.0E−07 | 5.0E−04 |
| r (nm) = | 1.4E−01 | 1.4E+00 | 4.5E+01 |

If the diffusion distance is comparable to or greater than R₀, diffusion should enhance energy transfer, particularly if the average separation between donor and acceptor is >R₀. The table shows that, for typical values of R₀ (e.g., 3-10 nm), DE-RET may be important for intermediate-lifetime donors and may be extremely important for long-lifetime donors (τ>1 μs, and especially τ>100 μs). Thus, lanthanides with their long lifetimes would be particularly suitable for DE-RET assays.

One application of DE-RET provided by the invention is to overcome a limitation in current RET assays caused by rigid binding of donor-acceptor pairs. For example, labeling often is indirect. The interaction between two biological molecules X and Y is followed by incubation of X and Y with D-P and S-A, where D is the donor, P interacts noncovalently with X, A is the acceptor, and S interacts noncovalently with Y. The final complex looks like:

D-P:X:Y:S-A

This arrangement is convenient for labeling (it often is easier to label P and S than to label X and Y directly); however, this arrangement can leave D and A widely separated, so that energy transfer is inefficient. In contrast, if the covalent linkages D-P and S-A are made long and flexible (e.g., without limitation, by using polyethoxy linkers), D and A can approach each other transiently by diffusion-like conformational fluctuations, enhancing energy transfer even though the covalent spacer has been lengthened.

Another application of DE-RET provided by the invention is in assays for X:Y binding involving the use of surface-labeled liposomes, in which the components are free to diffuse laterally in the plane of the lipid bilayer.

Liposomes may be labeled with A. One binding molecule could be attached to the liposome, and the other binding molecule could be attached directly or indirectly to D. Diffusion then will increase the probability that any given A will interact with a D (relative to the static situation).

Liposomes also may be labeled with D. Diffusion then will increase the probability that any given D will interact with an A (relative to the static situation). This will overcome a potential problem with particle-based static RET assays. Specifically, it generally is good to cover the surface of the particle with D's to maximize the probability of energy transfer to a small number of A's that may be bound to the liposome. However, if only if a small number of A's are present, the large background from the many D's that do not participate in RET will limit sensitivity. Diffusion effectively expands the interaction range of D's, permitting a smaller number to be used on the particle and decreasing the background at low levels of A binding.

D. Polarization Measurements With Biotinylated Ru-Encapsulated Macromolecules (Ru-MM-Biotin)

This section presents an application of the invention to polarization measurements involving biotinylated Ru-encapsulated macromolecules. Specifically, the luminophore 4,4′-dimethylcarboxy-2,2′bipyridine-bis(4,4′-diphenyl-2,2′-bipyridine) ruthenium (II) tetraphenylborate was encapsulated in beads (Ru-MM). The structure of the luminophore is shown below:

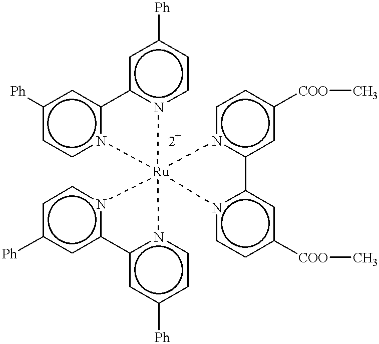

The beads were then reacted with biotin-NHS using ethylene diamine as a crosslinker.

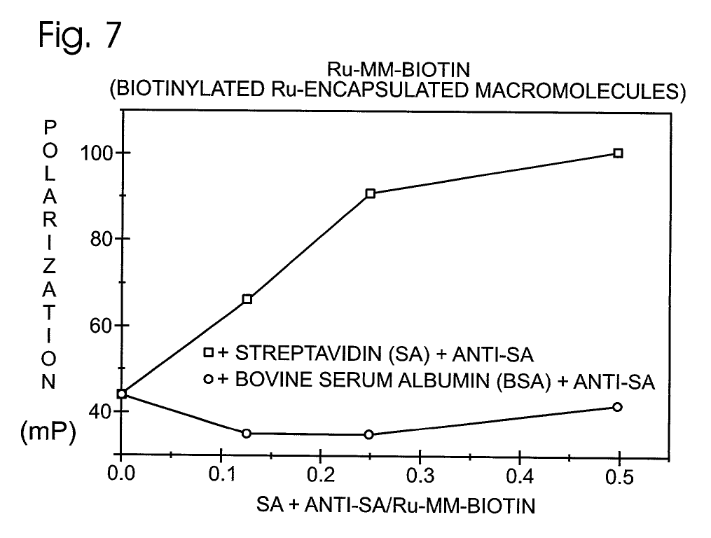

FIG. 7 shows results from polarization experiments conducted using these biotinylated-macromolecules (Ru-MM-biotin, MW˜20 kDa), which demonstrate that encapsulated polarization probes can be used to measure changes in rotational motions of larger proteins. Here biotinylated Ru-MM was first incubated with streptavidin (SA), followed by the addition of equivalent amounts of anti-SA. The polarization of the Ru-MM-biotin increased more than about twofold from 44 mP to about 100 microplate, while no change in polarization was observed in the control experiment using the same concentrations of bovine serum albumin (BSA) with anti-streptavidin.

The Ru-MM-biotin concentration chosen in the polarization measurement was 20 nM and the biotin concentration was 200 nM. Each macromolecule contained about 3-4 Ru-complexes, with an estimated dye/biotin ratio of 1:10.

The Ru-MM were prepared as follows. 6 mg EDC were added to 1.5 ml Ru-MM solution under constant stirring. Then 3.6 mg N-hydroxysuccinimide were added, and the solution was stirred at room temperature for 2 hours.

60 μL of ethylene diamine was dissolved in 300 μL of 100 mM sodium carbonate buffer at pH 8.9. The above carboxyl activated Ru-MM solution was added slowly into the ethylene diamine solution. The mixture was stirred at room temperature for 2 hours and then dialyzed for 17 hours against a 100 mM sodium carbonate buffer (pH 8.9) in a MWCO 3500 dialysis disk. Afterwards the Ru-MM solution was concentrated for 30 minutes using a Pierce concentrating solution kit.

3.2 mg Biotin-X SSE (6-[(biotinoyl)amino] hexanoic acid, sulfo NHS-LC-Biotin, Molecular Probes, Inc) was added to the above solution. The mixture was stirred at room temperature for 2.5 hours and then dialyzed for 15 hours against a 10 mM PBS buffer (pH 7.4) in a MWCO 3500 disk.

The biotin concentration was determined to be 120 μM using a Pierce Immunopure HABA solution and the Ru-complex concentration determined by absorption was 12 μM.

A similar procedure was used for biotinylation of the Ru-beads. The biotin concentration in the Ru-beads was determined to be 1 mM with an estimated Ru concentration of 24 μM.

Although the invention has been disclosed in its preferred forms, the specific embodiments thereof as disclosed and illustrated herein are not to be considered in a limiting sense, because numerous variations are possible. Applicants regard the subject matter of their invention as including all novel and nonobvious combinations and subcombinations of the various elements, features, functions, and/or properties disclosed herein. No single feature, function, element or property of the disclosed embodiments is essential. The following claims define certain combinations and subcombinations of features, functions, elements, and/or properties that are regarded as novel and nonobvious. Other combinations and subcombinations may be claimed through amendment of the present claims or presentation of new claims in this or a related application. Such claims, whether they are broader, narrower, equal, or different in scope from the original claims, also are regarded as included within the subject matter of applicants' invention.

We claim:

1. A method for detecting the presence or activity of an analyte in a sample, the method comprising:
forming a complex including first, second, and third members, where the first member is a probe, the second member is a mass label, and the third member is selected from the group consisting of the analyte, a compound that specifically binds to the analyte, and a product formed by the analyte, wherein no significant binding occurs involving the first and second members in the absence of the third member;
measuring a property of the probe that is sensitive to the size of the complex; and
correlating the property with the presence or activity of the analyte in the sample.

2. The method of claim 1, wherein the third member is the analyte.

3. The method of claim 1, wherein the third member is a receptor for the analyte.

4. The method of claim 1, wherein the third member is an enzyme.

5. The method of claim 1, wherein the probe is photoluminescent.

6. The method of claim 5, wherein the step of measuring a property of the probe includes the step of detecting a change in polarization.

7. The method of claim 5, wherein the photoluminescence lifetime of the probe is greater than the rotational correlation time of the unbound probe and less than the rotational correlation time of the complex formed by binding of the first, second, and third members.

8. The method of claim 1, wherein the probe binds to the third member noncovalently.

9. The method of claim 8, wherein the probe includes at least one of an immunological binding partner of the third member and a particulate.

10. The method of claim 1, wherein the mass label includes at least one of an immunological binding partner of the third member and a particulate.

11. The method of claim 1, wherein the mass label is capable of specifically binding to more than one third member.

12. The method of claim 1, the mass label being a first mass label, further comprising a second mass label capable of specifically binding to at least one third member, the complex formed by binding of the probe to the third member, and the mass label, but not to the probe alone.

13. The method of claim 12, wherein the second mass label is capable of specifically binding to at least two first mass labels, so that the second mass label may form crosslinks between third members.

14. The method of claim 12, wherein the second mass label includes at least one of the following: avidin, biotin, lectin, sugar, and an immunological binding partner.

15. The method of claim 1 further comprising the step of selecting the probe and mass label such that the average number of mass labels bound to third members exceeds the average number of probes bound to third members.

16. The method of claim 1, wherein the property of the probe is related to a rotational diffusion coefficient of the probe.

17. The method of claim 16, wherein the property of the probe is measured using a technique selected from the group consisting of polarization, light scattering, and magnetic resonance.

18. The method of claim 1, wherein the property of the probe is related to the translational diffusion coefficient of the probe.

19. The method of claim 1, the sample being a first sample, further comprising repeating with a second sample the steps of forming a complex, measuring a property of the probe, and correlating the property with the presence or activity of the analyte.

20. The method of claim 19 further comprising the step of comparing the amounts or activity of analyte in the first and second samples.

21. The method of claim 1, wherein the step of correlating the property with the presence or activity of analyte in the sample includes the step of quantifying the amount of analyte in the sample.

22. The method of claim 1 further comprising the step of correlating the presence of analyte with the presence or activity of another compound.

23. The method of claim 22, wherein the other compound is an enzyme.

24. The method of claim 1, wherein the step of correlating the property with the presence of analyte includes the step of comparing the property measured in the sample with the property measured in the absence of analyte.

25. The method of claim 1 further comprising the step of forming a complex includes the step of selecting the probe and mass label.

26. The method of claim 1, wherein the step of forming a complex includes the step of contacting the sample with the probe and mass label.

27. A kit for detecting the presence or activity of an analyte in a sample, the kit comprising:
a probe capable of specifically binding to a member, where the member is selected from the group consisting of the analyte, a compound that specifically binds to the analyte, and a product formed by the analyte; and
a mass label capable of specifically binding to the member or to a complex formed by binding of the probe to the member, but not to the probe alone;
wherein a measurable property of the probe is sensitive to the size of the complex formed by binding of the probe, member, and mass label.

28. The kit of claim 27, wherein the probe is photoluminescent.

29. The kit of claim 28, wherein the photoluminescence lifetime of the probe is greater than the rotational correlation time of the unbound probe and less than the rotational correlation time of the complex formed by binding of the probe, member, and mass label.

30. The kit of claim 27, wherein the probe binds to the member noncovalently.

31. The kit of claim 30, wherein the probe includes at least one of an immunological binding partner of the member and a particulate.

32. The kit of claim 27, wherein the mass label includes at least one of an immunological binding partner of the member and a particulate.

33. The kit of claim 27, wherein the mass label is capable of specifically binding to more than one member.

34. The kit of claim 27, the mass label being a first mass label, further comprising a second mass label capable of specifically binding to at least one of the member, the complex formed by binding of the probe to the member, and the first mass label, but not to the probe alone.

35. The kit of claim 34, wherein the second mass label is capable of specifically binding to at least two first mass labels, so that the second mass label may form crosslinks between members.

36. The kit of claim 34, wherein the second mass label includes at least one of the following: avidin, biotin, lectin, sugar, and an immunological binding partner.

37. The kit of claim 27, wherein the probe is not normally present in the sample.

38. The kit of claim 27, wherein the mass label is not normally present in the sample.

39. The kit of claim 27, wherein the property of the probe is related to a rotational diffusion coefficient of the probe.

40. The kit of claim 39, wherein the property may be measured using a technique selected from the group consisting of polarization, light scattering, and magnetic resonance.

41. The kit of claim 27, wherein the property of the probe is related to the translational diffusion coefficient of the probe.

42. A method of performing a resonance energy transfer assay, the method comprising:
providing first and second members of a donor/acceptor pair, the pair being capable of resonance energy transfer;
binding the first member to a binding partner;
permitting the first member to be diffusionally mobile relative to the binding partner while it is bound to the binding partner; and
detecting a change in proximity between the first and second member.

43. The method of claim 42, wherein the first member is associated with a first binding partner and the second member is associated with a second binding partner, and wherein the change in proximity between the first and second members is due to binding between the first and second binding partners.

44. The method of claim 42, wherein the first and second members are associated with a common substrate, and wherein the change in proximity between the first and second members is due to cleavage of the common substrate.

45. The method of claim 43, wherein the common substrate is a protein, and wherein the cleavage is effected by a protease.

46. The method of claim 43, wherein the common substrate is a nucleic acid, and wherein the cleavage is effected by a nuclease.

47. The method of claim 42, wherein the first member is diffusionally mobile across a surface.

48. The method of claim 47, wherein the surface is selected from the group consisting of a planar lipid bilayer, a liposome, and a cell membrane.

49. The method of claim 47, wherein the surface includes a plurality of first members.

50. The method of claim 42, wherein the first member is bound to the binding partner by a tether, the tether being sufficiently flexible that energy transfer between the first and second members is diffusionally enhanced.

51. The method of claim 42, wherein the step of detecting a change in proximity includes the steps of:
illuminating the members, so that the donor is excited;
detecting light emitted from at least one of the members; and
calculating the amount of energy transfer between the donor and acceptor based on the emitted light.

52. The method of claim 51, wherein the detected light is emitted by the donor.

53. The method of claim 51, wherein the detected light is emitted by the acceptor.

54. The method of claim 42, wherein the photoluminescence lifetime of the donor exceeds the photoluminescence lifetime of the acceptor.

55. The method of claim 42 further comprising the step of correlating the change in proximity with the presence of an analyte in a sample.

56. The method of claim 55, the sample being a first sample, further comprising repeating the steps of providing, binding, permitting, and detecting using a second donor/acceptor pair, and correlating the change in proximity with the presence of analyte in a second sample.

57. The method of claim 42 further comprising the steps of binding the second member to a second binding partner, and permitting the second member to be diffusionally mobile relative to the second binding partner while it is bound to the second binding partner.

58. A composition of matter comprising:
a particulate, and
a luminophore associated with the particulate,
wherein the size of the particulate and the lifetime of the luminophore are selected so that depolarization of the luminophore is detectable in a polarization assay.

59. The composition of claim 58, wherein the particulate is selected from the group consisting of a macromolecule, a dendrimer, a glass bead, a latex bead, a polyacrylnitrile bead, and a liposome.

60. The composition of claim 58, wherein the luminophore is encapsulated in the particulate.

61. The composition of claim 58, wherein the luminophore is covalently bound to the particulate.

62. The composition of claim 58, wherein the luminophore is entrapped in the particulate.

63. The composition of claim 58, wherein the luminophore is a metal-ligand complex.

64. The composition of claim 58, wherein the luminophore is coupled to the particulate so that the luminophore retains a fixed orientation relative to the particulate.

65. The composition of claim 58, wherein the luminophore is Ru-trix-bathophenanthroline.

66. The composition of claim 58, wherein the size of the particulate and the lifetime of the luminophore are selected so that luminescence emitted from free particulate bound to luminophore in a luminescence polarization assay is substantially unpolarized.

67. The composition of claim 58, wherein the particulate is labeled with at least one ligand.

68. The composition of claim 58, wherein the particulate is labeled with multiple ligands.

69. A method of detecting the presence or activity of a molecule of interest in a sample, the method comprising:
providing a particulate associated with a luminophore wherein the lifetime of the luminophore is long enough relative to the size of the particulate so that luminescence emitted from the luminophore is significantly depolarized,
contacting the particulate and luminophore with a sample containing the molecule of interest,
detecting a change in polarization of luminescence emitted by the luminophore, and
correlating the change in polarization with a property of the molecule of interest.

70. The method of claim 69 further comprising the step of selecting a size of the particulate and a lifetime of the luminophore so that luminescence emitted from free particulate bound to luminophore in a luminescence polarization assay is substantially unpolarized.

71. The method of claim 69 further comprising the step of encapsulating the luminophore in the particulate.

72. The method of claim 69 further comprising the step of entrapping the luminophore in the particulate.

73. The method of claim 69 further comprising the step of covalently binding the luminophore to the particulate.

74. The method of claim 69 further comprising the step of labeling the particulate with at least one ligand.

75. The method of claim 69 further comprising the step of agglutinating multiple particulates in the presence of the molecule of interest.

76. A method of performing a resonance energy transfer assay, the method comprising:
providing first and second members of a resonance energy transfer pair, wherein the first member is associated with a particulate,
contacting a sample with the resonance energy transfer pair under conditions such that proximity of the first member relative to the second member indicates a property of a molecule of interest.

77. The method of claim 76, wherein the providing step includes the step of associating the first member with a particulate selected from the group consisting of a macromolecule, a dendrimer, a glass bead, a latex bead, a polyacrylnitrile bead, and a liposome.

78. The method of claim 76, wherein the providing step includes the step of encapsulating the first member in the particulate.

79. The method of claim 76, wherein the providing step includes the step of entrapping the first member in the particulate.

80. The method of claim 76, wherein the providing step includes the step of covalently binding the first member with the particulate.

81. The method of claim 76, wherein the providing step includes the step of associating plural first members with a single particle.

82. The method of claim 76, wherein the property of the molecule of interest may be quantitative presence, qualitative presence, or activity.
